(19) United States
(12) Patent Application Publication
(10) Pub. No.: US 2022/0165376 A1
(43) Pub. Date: May 26, 2022

(54) INFORMATION PROCESSING APPARATUS, INFORMATION PROCESSING METHOD, AND INFORMATION PROCESSING PROGRAM

(71) Applicant: SONY GROUP CORPORATION, TOKYO (JP)

(72) Inventors: HARUHIKO YATA, TOKYO (JP); MIKI TOKITAKE, TOKYO (JP); TAIZO SHIRAI, TOKYO (JP)

(21) Appl. No.: 17/595,029

(22) PCT Filed: Apr. 24, 2020
(86) PCT No.: PCT/JP2020/017708

§ 371 Date: Nov. 5, 2021

(30) Foreign Application Priority Data
May 14, 2019 (JP) 2019-091516

(51) Int. Cl. G16H 10/60; G06F 3/01

(52) U.S. Cl. CPC G16H 10/60; G06F 2203/011; G06F 3/011

(57) ABSTRACT

Provided is an information processing apparatus including: an information acquisition unit that acquires genome information and related information regarding a subject; and an emotion estimation unit that estimates an emotion of the subject on the basis of the genome information and the related information.

### TECHNICAL FIELD

The present technology relates to an information processing apparatus, an information processing method, and an information processing program.

### BACKGROUND ART

In recent years, a technology of estimating a user's liking and preference, emotion, or the like and presenting content (movies, music, games, television (TV) programs, and the like) suitable for this user has been proposed.

Thus, there has been proposed a reproduction method capable of selecting and designating content reflecting an emotional or psychological state of a user using biometric information of a viewer/listener at the time of viewing and listening content of voice, music, and/or pictures or an analysis result for the biometric information (Patent Document 1).

### CITATION LIST

### Patent Document

Patent Document 1:
Japanese Patent Application Laid-Open No. 2004-246535

### SUMMARY OF THE INVENTION

### Problems to be Solved by the Invention

However, since the accuracy of the emotion estimation technology including the technology described in Patent Document 1 is not perfect yet, there is a disadvantage that there is a possibility that erroneous emotion determination occurs and content that does not fit the user's emotion is presented.

The present technology has been made in view of such a point, and an object thereof is to provide an information processing apparatus, an information processing method, and an information processing program capable of boosting the accuracy of emotion estimation.

### Solutions to Problems

In order to solve the above-described problem, a first technology is an information processing apparatus including: an information acquisition unit that acquires genome information and related information regarding a subject; and an emotion estimation unit that estimates an emotion of the subject on the basis of the genome information and the related information.

Furthermore, a second technology is an information processing method including: acquiring genome information and related information regarding a subject; and estimating an emotion of the subject on the basis of the genome information and the related information.

In addition, a third technology is an information processing program for causing a computer to execute an information processing method including: acquiring genome information and related information regarding a subject; and estimating an emotion of the subject on the basis of the genome information and the related information.

### BRIEF DESCRIPTION OF DRAWINGS

FIG. 1 is a block diagram illustrating external configurations of an information providing apparatus 100.

FIG. 2 is a block diagram illustrating a configuration of the information providing apparatus 100.

FIG. 3 is a block diagram illustrating a configuration of an information processing apparatus 200.

FIG. 4 is a flowchart illustrating processing in the information processing apparatus 200.

FIG. 5 is a flowchart illustrating response resolution processing.

FIG. 6 is a flowchart illustrating response resolution processing.

FIG. 7 is a flowchart illustrating response resolution processing.

FIG. 8 is a flowchart illustrating response resolution processing.

FIG. 9 is a flowchart illustrating response resolution processing.

FIG. 10 is a flowchart illustrating response resolution processing.

FIG. 11 is a flowchart illustrating response resolution processing.

FIG. 12 is a flowchart illustrating an application example of an information bank.

FIG. 13 is a flowchart illustrating an application example of an information bank.

FIG. 14 is a flowchart illustrating an application example of an information bank.

FIG. 15 is a block diagram illustrating a modification of the information processing apparatus 200.

FIG. 16 is a block diagram illustrating a modification of the information processing apparatus 200.

### MODE FOR CARRYING OUT THE INVENTION

Hereinafter, embodiments of the present technology will be described with reference to the drawings. Note that the description will be given in the following order.

<1. Embodiments>

[1-1. Configuration of Information Providing Apparatus 100]

[1-2. Configuration of Information Processing Apparatus 200]

[1-3. Regarding Genome Information and Epigenome Information]

[1-4. Processing in Information Processing Apparatus 200]

[1-5. Specific Example of Content Provision Using Information Processing Apparatus 200]

[1-6. Use of Information Bank]

<2. Modifications>

### 1. Embodiments

[1-1. Configuration of Information Providing Apparatus 100]

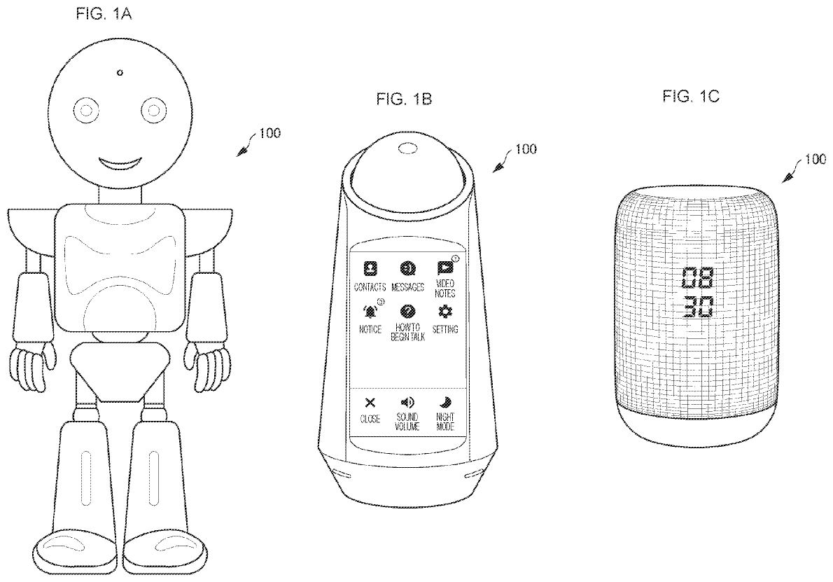

First, a configuration of an information providing apparatus 100 having the functions of an information processing apparatus 200 according to the present technology will be described. The information processing apparatus 200 works in the information providing apparatus 100. The information providing apparatus 100 is a so-called communication robot as illustrated in FIG. 1A, a so-called smart speaker as illustrated in FIGS. 1B and 1C, or the like capable of providing various sorts of information to a user in response to a request from the user or voluntarily. Furthermore, the information providing apparatus 100 may be a smartphone, a personal computer, a tablet terminal, wearable equipment, various sorts of Internet of Things (IoT) equipment, or the like.

Information provided to the user by the information providing apparatus 100 includes the user's schedule corresponding to the calendar, information about past events and incidents relating to the user, messages such as e-mails received by the user, notifications of various social network services (SNSs), and any other information that can be acquired through the Internet, such as weather, traffic information, and store information on restaurants. Furthermore, the information providing apparatus 100 is capable of replying, answering, and responding to verbal question, inquiry, or talk from the user by voice. Moreover, the information providing apparatus 100 is also capable of reproducing content such as music and pictures.

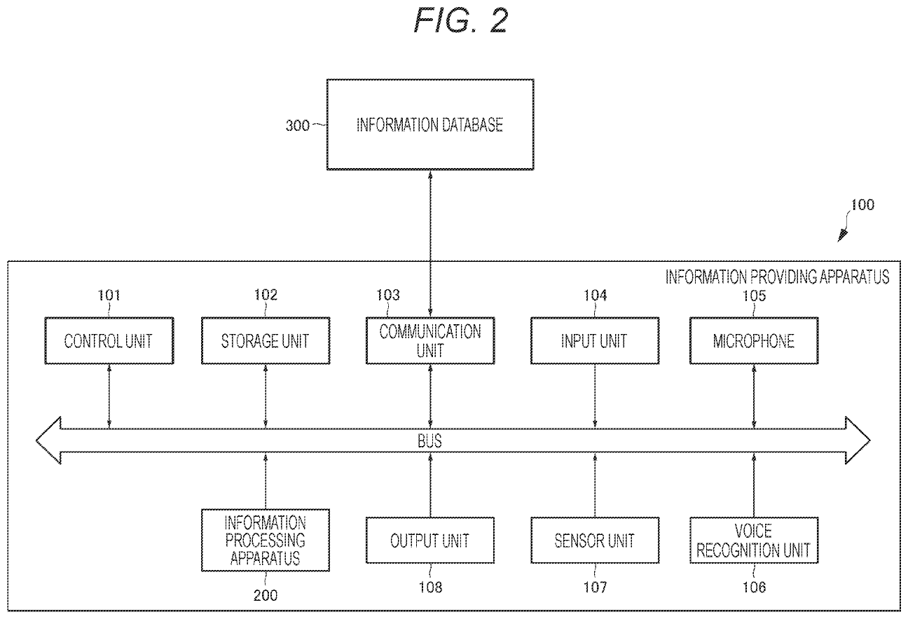

As illustrated in FIG. 2, the information providing apparatus 100 includes a control unit 101, a storage unit 102, a communication unit 103, an input unit 104, a microphone 105, a voice recognition unit 106, a sensor unit 107, an output unit 108, and the information processing apparatus 200.

The control unit 101 is constituted by a central processing unit (CPU), a random access memory (RAM), a read only memory (ROM), and the like. The CPU controls the entire information providing apparatus 100 and each unit by executing a variety of pieces of processing in accordance with a program stored in the ROM to issue a command.

The storage unit 102 is, for example, a mass storage medium such as a hard disk or a flash memory. The storage unit 102 holds various applications used by the information providing apparatus 100, various sorts of information input to the information providing apparatus 100 by the user, and the like.

The communication unit 103 is achieved by a communication module for transmitting and receiving data to and from the Internet, another apparatus, and the like. Any method of communication including a wireless local area network (LAN), a wide area network (WAN), wireless fidelity (WiFi), a fourth generation mobile communication system (4G), and broadband may be used as long as the method allows a connection to the Internet, another apparatus, and the like. Furthermore, the information providing apparatus 100 having functions as the information processing apparatus 200 communicates with an information database 300 through a network by the communication unit 103 to acquire genome information, epigenomes, other related information, and the like, and supplies the acquired information to the information processing apparatus 200.

The input unit 104 is for the user to input various instructions to the information providing apparatus 100. When an input is made to the input unit 104 by the user, a control signal according to the input is generated and supplied to the control unit 101. Then, the control unit 101 performs various pieces of processing corresponding to the supplied control signal. The input unit 104 may be a touch panel, a touch screen integrally configured with a monitor, or the like, as well as physical buttons.

The microphone 105 records a voice around the information providing apparatus 100 and supplies the recorded voice to the voice recognition unit 106 as an input voice signal. Normally, since information input and a request from the user to the information providing apparatus 100, which is a smart speaker, a communication robot, or the like, are made by an utterance, the microphone 105 collects the utterance from the user.

The voice recognition unit 106 analyzes the utterance of the user input from the microphone 105 using an existing voice recognition algorithm to recognize input contents, information, requests, and the like from the user. The recognized information and requests are supplied to the information processing apparatus 200.

The sensor unit 107 is a sensor that detects various sorts of information by sensing. The sensor includes an acceleration sensor, an angular velocity sensor, a geomagnetic sensor, an illuminance sensor, a temperature sensor, a humidity sensor, a barometric pressure sensor, and the like. For example, in a case where an apparatus including the sensor is carried or worn by the user, the above-described various sensors can detect various sorts of information as information relating to the user, for example, as information indicating a motion, a direction, or the like of the user. Furthermore, the sensor unit 107 may further include a sensor that detects biometric information of the user, such as pulse, perspiration, electroencephalogram, blood flow, tactile sensation, olfactory sensation, taste sensation, fingerprint, voiceprint, or vein. The input unit 104 may include a processing circuit that acquires information indicating the user's emotion by analyzing the information detected by these sensors and/or image or voice data detected by a camera or a microphone to be described later. Alternatively, the above information and/or data may be output to the information processing apparatus 200 without being subjected to analysis to be used for processing.

Moreover, the sensor may acquire an image or a voice in the vicinity of the user or the apparatus as data by a camera, a microphone, the above-described various sensors, or the like. In addition, the sensor may include a position detection means that detects an indoor or outdoor position. Specifically, the position detection means can include a global navigation satellite system (GNSS) receiver, for example, a global positioning system (GPS) receiver, a global navigation satellite system (GLONASS) receiver, or a BeiDou navigation satellite system (BDS) receiver, and/or a communication apparatus. The communication apparatus detects the position by utilizing a technology such as Wi-Fi (registered trademark), multi-input multi-output (MIMO), cellular communication (for example, location detection using a portable base station or femtocell), near field wireless communication (for example, Bluetooth low energy (BLE) or Bluetooth (registered trademark)), or low power wide area (LPWA). The sensor unit 107 is not restricted to the above sensors and may be any sensor as long as the sensor can detect data.

The output unit 108 is an output device for providing information to the user. The output unit 108 includes a display or a monitor that displays an image, a picture, a graphical user interface (GUI), and the like, a speaker that outputs voice, a light emitting diode (LED) that indicates information by turning on light, and the like.

The information providing apparatus 100 is configured as described above. The information providing apparatus 100 can acquire information by recognizing an utterance from the user by the voice recognition unit 106 and make a variety of responses according to the request of the user.

When accepting a request for information provision from the user, the information providing apparatus 100 makes a search on own information retained in the storage unit 102, a search on the Internet, or the like and, in a case where information that meets the request is acquired, provides the information to the user by an output from the output unit 108. Furthermore, the information providing apparatus 100 can provide the user with information input by the user and preserved, in response to a request from the user. In a case where information that meets the information provision request from the user is not successfully acquired, the information providing apparatus 100 notifies the user that the information has not been successfully acquired, by a message such as “No information” or “I have no idea”.

[1-2. Configuration of Information Processing Apparatus 200]

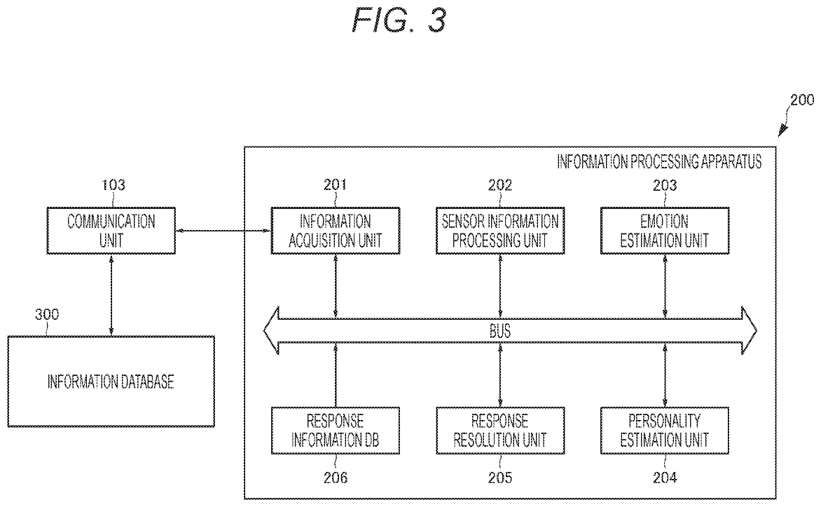

A configuration of the information processing apparatus 200 according to the present technology will be described with reference to FIG. 3. Note that the information processing apparatus 200 can acquire information from the external information database 300 to individually perform emotion estimation and personality estimation and utilize the information for emotion estimation, personality estimation, and response content resolution for a person to which a response is to be output by the information providing apparatus 100 (hereinafter referred to as a subject).

The information processing apparatus 200 has a configuration including an information acquisition unit 201, a sensor information processing unit 202, an emotion estimation unit 203, a personality estimation unit 204, a response resolution unit 205, and a response information database 206.

The information acquisition unit 201 acquires sensor information from the information providing apparatus 100 in which the information processing apparatus 200 works, or another external apparatus. Furthermore, genome information, epigenome information, other related information, and the like are acquired from the information database 300. The acquired information is supplied to the sensor information processing unit 202, the emotion estimation unit 203, the personality estimation unit 204, and the response resolution unit 205 from the information acquisition unit 201. Note that, in the present embodiment, information other than the genome information used for personality estimation, emotion estimation, and response resolution is defined as related information regarding the subject.

The sensor information processing unit 202 conducts diverse types of processing on the sensor information supplied from the sensor unit 107 and, for example, extracts a feature amount. Note that the sensor information supplied to the information processing apparatus 200 may be not only the sensor information detected by the sensor unit 107 included in the information providing apparatus 100 but also sensor information detected by another external sensor apparatus or apparatus having a sensor function. For example, a smartphone, a wearable device, or the like used by the subject may be employed. Furthermore, a facility capable of detecting biometric information or the like may detect the biometric information and supply the detected biometric information to the information processing apparatus 200 via the Internet, for example.

The emotion estimation unit 203 estimates an emotion of the subject on the basis of a feature point extracted by the sensor information processing unit 202, and the genome information, epigenome information, and other related information acquired from the information database 300. For example, the emotion estimation method includes a well-known method of performing estimation on the basis of biometric information such as heartbeat, pulse wave, respiration, blood pressure, electrocardiogram, electroencephalogram, skin perspiration, skin resistance, body movement, posture, magneto-encephalogram, myoelectric potential, body surface temperature, size of pupil diameter, microvibration, nictitation (blinking), and biochemical reaction of the subject. Furthermore, there is also a well-known method of estimating the emotion from the subject's voice, secretion, expression information on the subject that can be acquired from an image obtained by capturing the subject, and the like. In addition, the emotion estimation unit 203 can also estimate the emotion on the basis of action history information of the subject and environmental information around the subject as the related information. Moreover, the emotion estimation unit 203 can also estimate the emotion on the basis of a past exchange history between the information providing apparatus 100 and the subject.

The emotion estimation unit 203 estimates the emotion of the subject using the genome information, the epigenome information, and other related information, in addition to the well-known emotion estimation methods described above.

The personality estimation unit 204 estimates the personality of the subject using the genome information, the epigenome information, and other related information, in addition to well-known personality estimation methods. Note that, since the personality of a person is less likely to change depending on the environment or the like and does not change significantly in a lifetime, the personality can be estimated mainly on the basis of the genome information.

The present technology enables highly accurate personality estimation and emotion estimation by using the genome information and the epigenome information in addition to related information such as biometric information, image information, and voice information, which have been conventionally used for emotion estimation and personality estimation.

The response resolution unit 205 resolves various contents of responses (provision of content, a response to a request from the subject, and the like) of the information providing apparatus 100 to the subject, on the basis of an emotion estimation result, a personality estimation result, and information acquired from the information database 300. The processing in the response resolution unit 205 varies depending on what apparatus the information processing apparatus 200 works in.

The response information database 206 holds a large number of response patterns of the information providing apparatus 100 according to the request from the subject and the state of the subject, and is referred to when the response resolution unit 205 resolves the response contents.

The information processing apparatus 200 is constituted by a program. This program may be installed in the information providing apparatus 100 in advance or may be distributed by download, a storage medium, or the like to be installed personally by the user. Note that the information processing apparatus 200 may be not only implemented by a program but also implemented by combining a dedicated apparatus, circuit, and the like by hardware having the function of the program.

The information processing apparatus 200 may start the processing by being triggered by voice input to the information providing apparatus 100, or may also start the processing by being triggered by the expression of the face of the subject detected from an image captured by the camera, apart from the voice input. Moreover, the processing may be started by being triggered by the detection of the above-described various sorts of information by the sensor unit 107.

Furthermore, the information processing apparatus 200 may work not in the information providing apparatus 100 but in a server, a cloud, another apparatus apart from the information providing apparatus 100, or the like to transmit the emotion estimation result, the personality estimation result, the resolved response contents, and the like to the information providing apparatus 100. Moreover, the emotion estimation result, the personality estimation result, the resolved response contents, and the like may be transmitted to an apparatus other than the information providing apparatus 100 while being transmitted to the information providing apparatus 100.

The information database 300 is, for example, a database referred to as an information bank. The information bank (information utilization credit bank) is a business that manages personal data by exploiting a system such as a personal data store (PDS) on the basis of a contract or the like with an individual relating to data exploitation, and also provides data to a third party (another business operator) after verifying validity on behalf of the individual on the basis of an instruction from the individual or a condition designated in advance.

In order to appropriately use the information providing apparatus 100 and the information processing apparatus 200, the subject needs to preserve the own genome information, epigenome information, and other related information in the information database 300 in advance. Then, these pieces of information are provided to the information processing apparatus 200 via a network as necessary.

[1-3. Regarding Genome Information and Epigenome Information]

Here, the genome information and the epigenome information will be described. The genome is base sequence information on deoxyribonucleic acid (DNA) and is genetic information on all nucleic acids possessed by an organism. Furthermore, the genome can also be referred to as an assortment obtained by putting together only one series of chromosomes from among all the chromosomes possessed by a certain organism, that is, all the chromosomes contained in a single-phase cell. A somatic cell of a normal organism is supposed to have two series of genomes. The genome of a human (person: Homo sapiens) is called the human genome and is one set of genetic information. The human genome is made up of a nuclear genome and a mitochondrial genome.

By analyzing the human genome, it is possible to know a relationship between human populations and individual's health state, disease risk, and the like. Regions of the human genome corresponding to extroversion, neurotic tendency, harmonicity, diligence, and openness, which are deemed to be five major factors of the personality of a person, have been specified, and the personality of a person can be estimated nowadays by examining the human genome. Moreover, it has also been found from the human genome that there is a correlation between personality and susceptibility to having a symptom of mental disease, such as “extroversion and attention-deficit hyperactivity disorder (ADHD)” and “neurotic tendency and depression”.

The epigenome is genetic information prescribed by chemical modifications to DNA and histones without a change in the base sequence of DNA and is a modification added to the genome. The epigenome is something that is developed and changes due to a developed environmental component by undergoing a change. Methylation and hydroxymethylation of DNA and modification to histone protein (methylation, acetylation, phosphorylation, and the like) are mainly known.

The epigenome is almost entirely passed on to the next new cell even when the cell divides. These chemical modifications are reversible and can be affected by external environments, meals, and the like. Although epigenomes are inherited across generations, individual differences and diversity have been reported from studies of identical twins and the like. The twins basically have the same genome, but do not have exactly the same appearance or vulnerability to illness. This is because the epigenome state of a human gradually changes as the human grows older. The variations in coat color and pattern between a clone cat and a cat that provided the genome are also due to the epigenome. For example, the pattern on the coat of a tri-color cat is chosen depending on not genomic genetic information but epigenome deviation. In plants, it is sad that the tie-dye pattern on the flowers of morning glory is chosen by epigenome. Furthermore, epigenome abnormalities are deeply related to human illness such as cancer, metabolic diseases, immune diseases, and gynecological diseases. In addition, hereafter, a person's favorite for food and preference in objects of interest. It is considered that the personality of a person, an emotion of a person, and the like can also be grasped from the epigenome.

Recent studies have revealed that, for example, genes affect emotions of a person, that specified races are genetically prone to have specified emotions, and that one third of emotions of a person are genetic elements while the remaining two thirds are external elements such as environments. Accordingly, it is considered that the personality, emotion, preference, and the like of the subject can be estimated on the basis of the genome information and the epigenome information of the subject.

In order to obtain the genome information and the epigenome information of the subject, it is necessary to take body tissue of the subject and perform well-known test, analysis processing, and the like. Since there are companies and organizations that perform genome test and analysis services, the genome information and the epigenome information of the subject can also be acquired using these companies and organizations. Therefore, a corporation that operates the information database 300 prompts the subject to undergo a test for acquiring the genome information and the epigenome information, and the genome information and the epigenome information provided from the subject are held and preserved in the information database 300. Alternatively, a corporation that operates the information database 300 prompts the subject to undergo a genome test and analysis service and is given the genome information and the epigenome information from a corporation or organization that performs the genome test and analysis service to hold and preserve the received information in the information database 300. In this manner, the genome information and the epigenome information of the subject are held and preserved in the information database 300. Note that the method of acquiring the genome information and the epigenome information of the subject is not restricted to these examples, and any method may be adopted as long as the information can be lawfully acquired.

Since the genome is unchanged, once the genome information of the subject is held in the information database 300, it is not necessary thereafter to acquire the genome information again and hold the genome information in the information database 300. On the other hand, since the epigenome keeps changing depending on the developed environment and the like, it is necessary to acquire the epigenome information of the subject regularly or irregularly and update the epigenome information held in the information database 300 in order to predict the personality and emotion more precisely.

For this reason, the business operator or the like that operates the information database 300 needs to regularly suggest the subject to provide the epigenome information. Furthermore, in a case where the epigenome information of the subject can be acquired at any time by a wearable device in the future, the epigenome information may be transmitted from the wearable device to the information database 300 constantly and continuously or regularly.

In addition, the information database 300 also holds information relating to each of a large number of human beings including the subject, as well as the genome information and the epigenome information. Such information includes a life log, detection information by a sensor, information indicating liking and preference, information obtained from use of an e-mail or an SNS, and the like. These pieces of information are preferably transmitted to the information database 300 regularly or irregularly to update databases in the information database 300. Precise emotion estimation can be performed by always keeping the state of the information in the information database 300 up to date.

Furthermore, the information database 300 may hold the genome information and the epigenome information of a third party apart from the subject for emotion estimation. This is because, in a case where such a third party has a relationship in various bodies with the subject of the emotion estimation and is present around the subject when the information processing apparatus 200 estimates the emotion, the information on the third party affects the result of emotion estimation for the subject in some cases.

[1-4. Processing in Information Processing Apparatus 200]

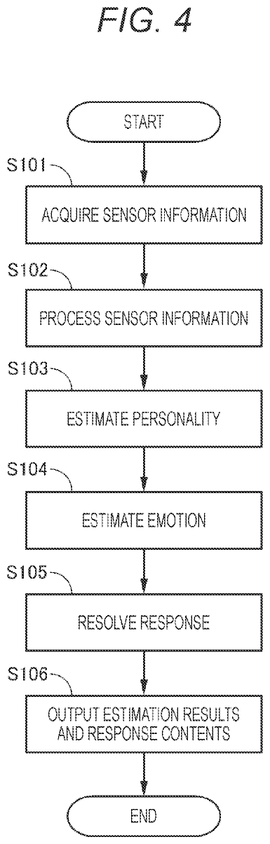

Next, processing in the information processing apparatus 200 will be described with reference to the flowchart in FIG. 4. First, in step S101, the information acquisition unit 201 acquires the genome information, the epigenome information, and other related information supplied from the information database 300 and additionally acquires sensor information and the like supplied from the information providing apparatus 100 or another external apparatus. The acquired various sorts of information are supplied to each of the sensor information processing unit 202, the personality estimation unit 204, the emotion estimation unit 203, and the response resolution unit 205.

Next, in step S102, the sensor information processing unit 202 conducts predetermined processing on the sensor information among the various sorts of information supplied from the information acquisition unit 201 and, for example, extracts a feature point. The extracted feature point information is supplied to the personality estimation unit 204, the emotion estimation unit 203, and the response resolution unit 205.

Next, in step S103, the personality estimation unit 204 estimates the personality of the subject on the basis of the genome information, the related information, the feature point information, and the like. Note that, normally, since the personality of a person does not change significantly even with the lapse of time or a change in environment, in a case where the information processing apparatus 200 once estimates the personality of the subject, the personality estimation may be omitted thereafter, and the already estimated personality estimation result may be used.

Next, in step S104, the emotion estimation unit 203 estimates an emotion of the subject on the basis of the genome information, the related information, the feature point information, and the like. Note that step S103, the personality estimation, and the emotion estimation in step S104 may be performed in the reverse order and are not limited to any one of the orders. The personality estimation processing may be performed first, or both pieces of the processing may be performed in parallel.

Next, in step S105, the response resolution unit 205 resolves the response contents of the information providing apparatus 100 to the subject on the basis of the genome information, the related information, the feature point information, the personality estimation result, the emotion estimation result, and the like.

Then, information indicating the resolved response contents is output to the information providing apparatus 100 in step S106. Subsequently to this, the information providing apparatus 100 makes a response to the subject. Furthermore, information indicating the personality estimation result and the emotion estimation result may be output as necessary.

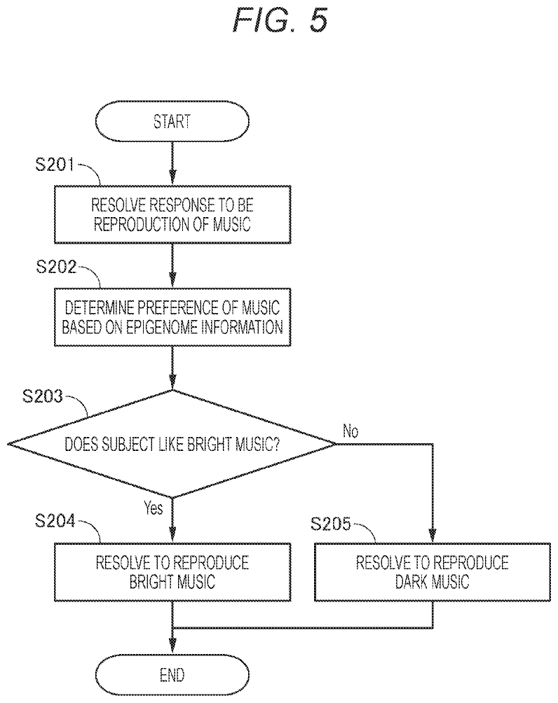

Here, details of the response resolution processing in step S105 in FIG. 4 will be described with reference to the flowchart in FIG. 5. In the response resolution processing, a variety of response contents are resolved from the personality estimation result, the emotion estimation result, the genome information, the epigenome information, and other related information of the subject.

First, as illustrated in FIG. 5, in a case where the response resolution unit 205 resolves the response contents to be “reproduction of music”, the response resolution unit 205 can further resolve the type of music to reproduce on the basis of the epigenome information.

In a case where the response resolution unit 205 resolves in step S201 that the response is to reproduce music, next, in step S202, the subject's preference of music is determined from the epigenome information.

In a case where it is determined from the epigenome information that the subject likes bright music (pleasant music), the processing proceeds to step S204 (Yes in step S203), and the response resolution unit 205 resolves to reproduce the bright music (pleasant music) as a response.

On the other hand, in a case where it is determined from the epigenome information that the subject does not like bright music (pleasant music), the processing proceeds to step S205 (No in step S203), and the response resolution unit 205 resolves to reproduce dark music (sad music) as a response.

Note that whether the music is bright (pleasant) or dark (sad) can be verified by analysis based on the tempo, tone, lyrics, and the like of the music. In order to make such a response, it is preferable for the information providing apparatus 100 or the information processing apparatus 200 to retain music data included in the information providing apparatus 100 or an analysis result for music that can be reproduced on the Internet, beforehand as a database.

Note that it is widely known that the preference of music varies depending on the epigenome even for an identical twin having the same DNA. Therefore, by using the epigenome information to resolve the response contents, response contents that further meet the preference of the subject can be resolved.

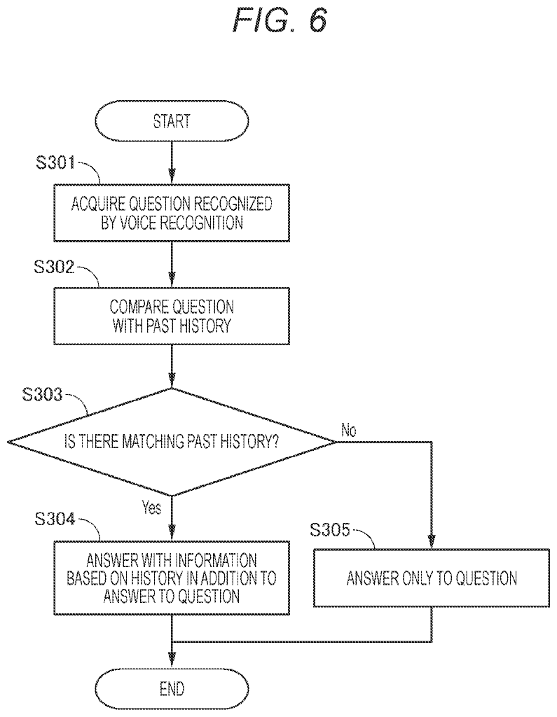

Furthermore, as illustrated in FIG. 6, for example, in a case where a question is put to the information providing apparatus 100 by the subject, the response resolution unit 205 can also resolve the response contents on the basis of a past history of exchanges between the information providing apparatus 100 and the subject.

First, in step S301, the information acquisition unit 201 acquires information indicating a question from the subject, which has been recognized by the voice recognition unit 106 included in the information providing apparatus 100. Next, in step S302, the response resolution unit 205 compares history information on past questions from the subject and responses of the information providing apparatus 100 to the past questions with the question from the subject at the current moment, and determines whether or not there is a matching past history. For example, this matching can be determined on the basis of a keyword or the like in the question from the subject.

In a case where there is a matching past history, the processing proceeds to step S304 (Yes in step S303), and the response resolution unit 205 resolves a response so as to also provide information that can be acquired from the past history together with an answer to the question.

On the other hand, in a case where there is a matching past history, the processing proceeds to step S305 (Yes in step S303), and the response resolution unit 205 resolves the response contents so as to output only an answer to the question.

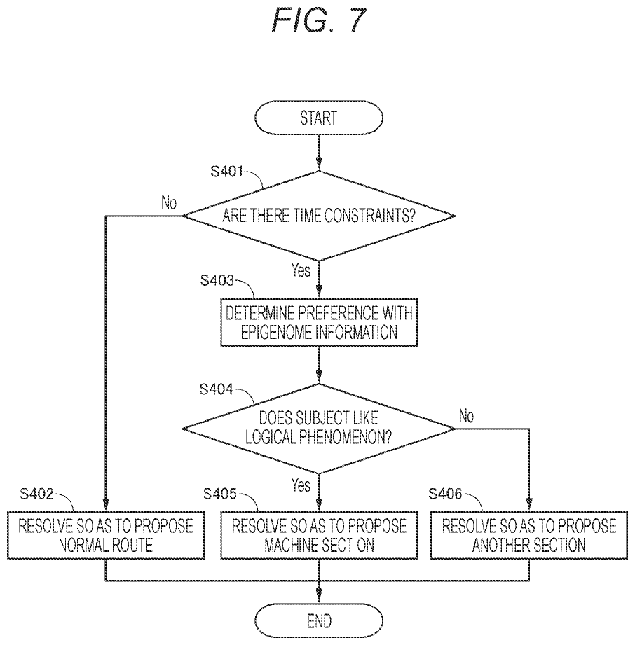

Furthermore, in a case where the information providing apparatus 100 proposes an action of the subject in a facility (for example, a museum or an art gallery), as illustrated in FIG. 7, the information processing apparatus 200 can resolve the proposal contents on the basis of the epigenome information of the subject. The proposal of the action of the subject means which section is preferentially guided to the subject in a case where the facility has a plurality of exhibit sections. In the example in FIG. 7, it is assumed that the information providing apparatus 100 confirms to the subject whether or not there are time constraints on the action of the subject.

First, in step S401, in a case where there is no time constraint, the processing proceeds to step S402 (Yes in step S401), and the response resolution unit 205 resolves the response contents so as to propose a normal route without proposing a specified section. This is because there is no time constraint on the action of the subject, and thus it is not necessary to preferentially propose a specified section.

In a case where there are time constraints, the processing proceeds to step S403 (Yes in step S401), and the preference of the subject is determined on the basis of the epigenome information of the subject. Here, as an example of the preference determination, it is supposed that “whether or not the subject likes a logical phenomenon” is determined.

In a case where the subject likes a logical phenomenon, the processing proceeds to step S405 (Yes in step S404), and the response contents are resolved so as to propose a machine section. Note that the machine section is merely an example of a matter that a person who likes logical phenomena is considered to prefer.

On the other hand, in a case where the subject does not like a logical phenomenon, the processing proceeds to step S406 (No in step S404), and the response contents are resolved so as to propose a section other than the machine section.

It is merely an example to propose the machine section in a case where it is determined that the subject likes a logical phenomenon on the basis of the epigenome information described above, and a variety of other applications are possible.

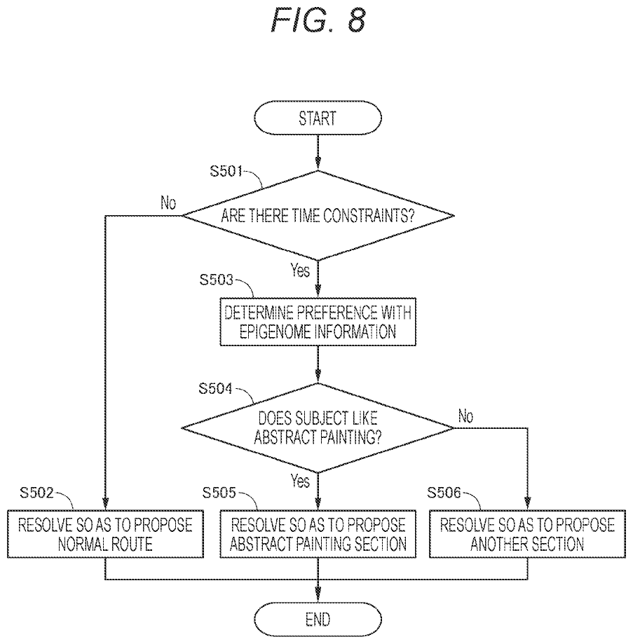

Next, another example of the proposal for the action of the subject in a facility will be indicated with reference to the flowchart in FIG. 8. First, in step S501, in a case where there is no time constraint, the processing proceeds to step S502 (Yes in step S501), and the response resolution unit 205 resolves the response contents so as to propose a normal route. This is because there is no time constraint on the action of the subject, and thus it is not necessary to preferentially propose a specified section.

In a case where there are time constraints, the processing proceeds to step S503 (Yes in step S501), and the preference of the subject is determined on the basis of the epigenome information. Here, as an example of the preference determination, it is supposed that “whether or not the subject likes an abstract painting” is determined.

In a case where the subject likes an abstract painting, the processing proceeds to step S505 (Yes in step S504), and the response resolution unit 205 resolves the response contents so as to propose an abstract painting section.

On the other hand, in a case where the subject does not like a logical phenomenon, the processing proceeds to step S506 (No in step S404), and the response resolution unit 205 resolves the response contents so as to propose a section other than the abstract painting section.

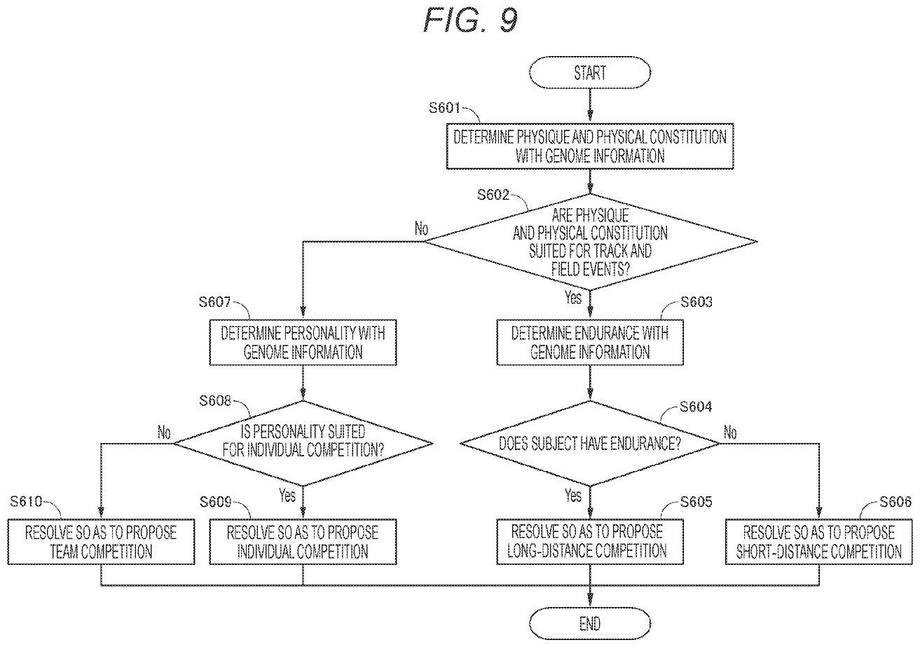

Furthermore, as illustrated in FIG. 9, it is also possible to propose sports to the subject on the basis of the genome information in addition to or instead of the epigenome information.

First, in step S601, the physique and the physical constitution of the subject are determined on the basis of the genome information of the subject. Since it is considered that physical features such as a person's physique and physical constitution are chosen by innate elements with which the person was born rather than developed elements, the determination can be made on the basis of the genome information.

In a case where the physique and the physical constitution of the subject are suited for the track and field events, the processing proceeds to step S603 (Yes in step S602). Next, in step S603, the response resolution unit 205 determines the endurance of the subject on the basis of the genome information. In a case where the endurance of the subject is equal to or higher than a predetermined reference, the processing proceeds to step S605 (Yes in step S604), and the response contents are resolved so as to propose a long-distance competition. This is a result derived from the fact that the subject is suited for the track and field events and has endurance.

On the other hand, in a case where the endurance is equal to or lower than the predetermined reference, the processing proceeds to step S606 (No in step S604), and the response contents are resolved so as to propose a short-distance competition. This is a result derived from the fact that the subject is suited for the track and field events but has no endurance.

The explanation returns to step S602. In a case where the physique and the physical constitution of the subject are not suited for the track and field events, the processing proceeds to step S607 (No in step S602). Whether or not the physique and the physical constitution of the subject are suited for the track and field events can be comprehensively determined from, for example, physical features such as the height and the lengths of arms and legs, the vital capacity, the muscle mass, and the like. Next, in step S602, the personality estimation unit 204 determines the personality of the subject on the basis of the genome information.

In a case where the personality of the subject is suited for the individual competition, the processing proceeds to step S609 (Yes in step S608), and the response contents are resolved so as to propose an individual competition. On the other hand, in a case where the personality of the subject is not suited for the individual competition, the processing proceeds to step S610 (No in step S608), and the response contents are resolved so as to propose a team competition.

Note that, as a method of determining whether or not the subject is suited for the individual competition from the personality, for example, in a case where the personality is verified to be selfish, unsociable, or the like, it can be determined that the subject is suited for the individual competition.

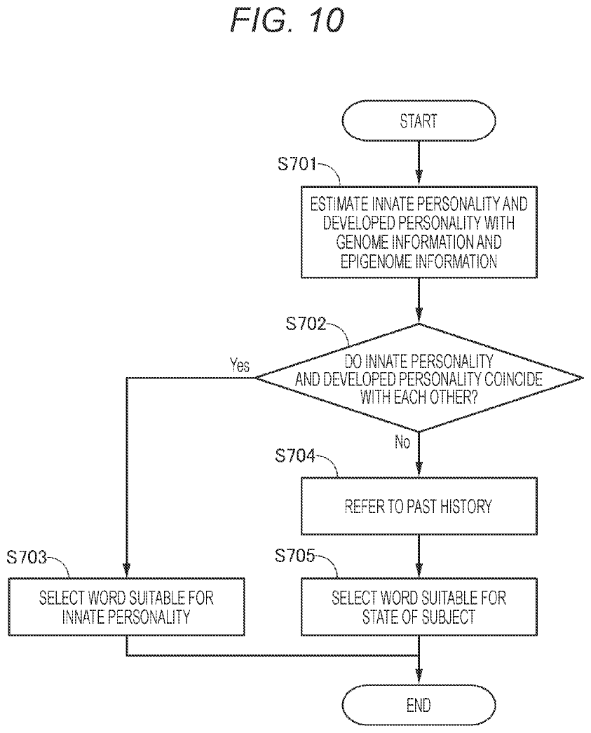

Moreover, a word suitable for the feelings of the subject to be output from the information providing apparatus 100 to the subject can be resolved using the genome information and the epigenome information as illustrated in FIG. 10.

First, in step S701, the personality estimation unit 204 estimates the innate personality and the developed personality of the subject on the basis of the genome information and the epigenome information.

Next, in step S702, the response resolution unit 205 determines whether or not the innate personality coincides with the developed personality. In a case where the innate personality coincides with the developed personality, the processing proceeds to step S703 (Yes in step S702). Then, in step S703, the response resolution unit 205 refers to the database, the Internet, and the like and resolves the response contents so as to output a word suitable for the innate personality of the subject.

On the other hand, in a case where the innate personality and the developed personality do not coincide with each other, the processing proceeds to step S704 (No in step S702). Next, in step S704, the response resolution unit 205 refers to the history of words output by the information providing apparatus 100 to the subject in the past.

Then, in step S705, the response resolution unit 205 resolves the response contents so as to output a word suitable for the current state of the subject (an emotion at the current moment estimated by the emotion estimation unit, or the like).

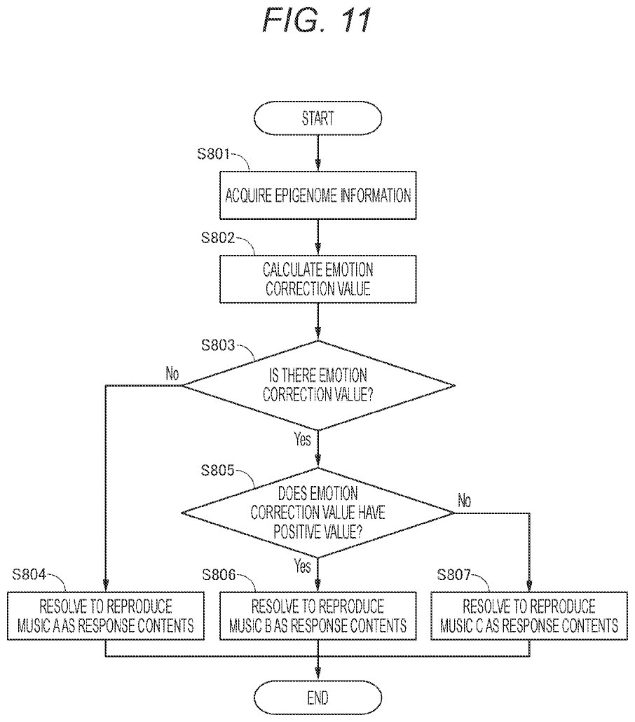

The emotion estimation unit 203 estimates an emotion of the subject on the basis of the genome information, the epigenome information, and other related information, and the response resolution unit 205 resolves the response contents to the subject on the basis of the estimated emotion. However, as illustrated in the processing in FIG. 11, for example, in a case where a factor that further emphasizes an emotion is found from the epigenome information, the response contents can be resolved so as to be suitable for this emphasized emotion. Furthermore, in a case where a factor that further suppresses an emotion is found from the epigenome information, the response contents can be resolved so as to be suitable for this suppressed emotion.

First, in step S801, the information acquisition unit 201 acquires the epigenome information of the subject from the information database 300 and supplies the acquired epigenome information to the response resolution unit 205. Next, in step S802, the response resolution unit 205 calculates a value that corrects the estimated emotion (referred to as an emotion correction value) on the basis of the epigenome information. This emotion correction value is, for example, calculated as a positive value in a case where the epigenome information has a large element that further emphasizes the emotion. On the other hand, the emotion correction value is calculated as a negative value in a case where the epigenome information has a large element that further suppresses the emotion.

Next, in step S803, the response resolution unit 205 verifies whether or not there is an emotion correction value. In a case where there is no emotion correction value, the processing proceeds to step S804 (No in step S803). Then, in step S804, the response resolution unit 205 resolves the response contents so as to reproduce music suitable for the already estimated emotion as a default.

On the other hand, in a case where there is an emotion correction value, the processing proceeds to step S805 (Yes in step S803). Next, in step S805, the response resolution unit 205 verifies whether or not the emotion correction value has a positive value. In a case where the emotion correction value has a positive value, the processing proceeds to step S806 (Yes in step S805). Then, in step S806, the response resolution unit 205 resolves the response contents so as to reproduce music suitable for a state in which the already estimated emotion is emphasized. For example, in a case where the subject's emotion is estimated to be “sad” and the response contents have been resolved so as to reproduce pleasant music as normal response resolution processing, it is selected to calm down the feelings with quiet music rather than encouraging the subject with pleasant music in a case where the subject's emotion is in a state in which being “sad” is emphasized with the emotion correction value.

On the other hand, in a case where the emotion correction value has a negative value, the processing proceeds to step S807 (No in step S805). Then, in step S807, the response resolution unit 205 resolves the response contents so as to reproduce music suitable for a state in which the already estimated emotion is restrained. For example, in a case where the subject's emotion is estimated to be “sad” and the response contents have been resolved so as to reproduce pleasant music as normal response resolution processing, it is selected to uplift the feelings with intense music rather than encouraging the subject with pleasant music in a case where the subject's emotion is in a state in which being “sad” is suppressed with the emotion correction value.

The processing in the information processing apparatus 200 is performed as described above. Note that, in the flowcharts illustrated in FIGS. 5 to 10, the response contents are resolved from among two or three options, but this is an example of the options indicated for convenience of description. In the present technology, it is considered that the response contents can be actually resolved with high accuracy from among options more than the above example, such as several tens of options, several hundreds of options, or more options, by using the genome information and the epigenome information.

[1-5. Specific Example of Content Provision Using Information Processing Apparatus 200]

The processing by the information processing apparatus 200 described above allows the information providing apparatus 100 having the functions of the information processing apparatus 200 to make a variety of responses to the subject. Specific examples thereof will be described.

First, in a case where it is estimated that a subject's emotion is “pleasant” when the subject makes a query regarding an incident in a specific past period, the information providing apparatus 100 presents information regarding a past incident classified as “pleasant” to the subject.

For this purpose, the information providing apparatus 100 needs to classify incidents of the subject in the past (for example, events registered in a calendar application) beforehand in correspondence with emotions. This emotion may be resolved on the basis of inputs from the subject, or may be estimated to be pleasant from the titles of the incidents. For example, predetermined incidents such as “movie” and “amusement park” are determined to be pleasant. Furthermore, in a case where the estimated emotion of the subject is “pleasant”, a smiling face may be detected from an image preserved in the information providing apparatus 100, and information regarding an incident in a period in which the image with the detected smiling face was captured may be presented to the subject.

In addition, in a case where there is a query regarding an incident in a specific past period from the subject, the estimated personality of the subject can also be employed as a verification material for resolving the response contents, and an answer to the question from the subject can be presented.

In addition, in a case where the emotion of the subject at present is estimated to be “sad”, the information providing apparatus 100 can reproduce music classified as “sad” for the subject.

In addition, in a case where the emotion of the subject can be estimated to be “bad mood” in addition to being “sad” when music is provided, the response contents may be resolved on the basis of extroversion, neurotic tendency, harmonicity, diligence, and openness, which are deemed to be five major factors of the personality in “human genome”, which is the whole gene information possessed especially by a human, among genomes. As a result, there can be a case where response contents of not reproducing (not presenting) music are given, for example.

Furthermore, in a case where the emotion of the subject is estimated to be “sad” and the personality of the subject is estimated to be “I want to be fine when there is a person near by” when there is a person around the subject, the information providing apparatus 100 can reproduce music classified as “pleasant” for the subject.

In addition, in a case where the emotion of the subject is estimated to be “sad” when there is a person around the subject and there is a predetermined relationship (such as a close relationship) between the subject and the person around the subject, information on an incident shared by the subject and the person around the subject can be presented. For example, the information on the incident shared by the subject and the person around the subject is the presentation of a photograph in which the subject and the person around the subject appear, or the like.

Note that an emotion and the personality of the person around the subject may be used for response resolution. If the emotion of the person around the subject is not taken into account, there can be a case where a response that deteriorates the relationship between the subject and the person around the subject is resolved. For example, the person around the subject can be specified by face authentication using an image captured by a camera included in the information providing apparatus 100, voiceprint authentication using a voice collected by a microphone, or the like. In a case where the person around the subject is specified by these methods, information on this specified human being is transmitted to the information database 300, and in a case where the information database 300 has the genome information, the epigenome information, and the like regarding this specified human being, these pieces of information are acquired from the information database 300.

Furthermore, in a case where the emotion of the subject is estimated to be “sad”, a search is made for a history of estimating the emotion of the subject to be sad in the past. In a case where it can be confirmed from the retrieved history that the subject has reproduced a specified music in the past sad time, it is also possible to reproduce the confirmed specified music.

The emotion estimation, personality estimation, and response resolution according to the present technology are performed as described above. According to the present technology, the accuracy of personality estimation and emotion estimation can be enhanced by using the genome information and the epigenome information in addition to the biometric information and the like conventionally used for emotion estimation and personality estimation. Furthermore, content that the subject wants now can be appropriately presented on the basis of the estimated precision and emotion. Therefore, it becomes also possible to personalize a variety of pieces of content such as a movie, an animation, and a game to every single individual, and to enjoy the provision of content suitable for the individual's own preference and content corresponding to an emotion at that time without the presentation of content that does not fit the emotion. In addition, even in a case where already personalized content such as a diary is read back according to the feelings or forgotten things are desired to be recalled, it is possible to prevent a past that is not desired to be recalled from being presented to hurt the feelings. Moreover, a Cobby robot or the like that can make an answer in a manner to discern one's mind in the one's own past and upcoming searches can also be implemented.

[1-6. Use of Information Bank]

Next, in a case where the information database 300 is an information bank, an example of use of the information bank will be described.

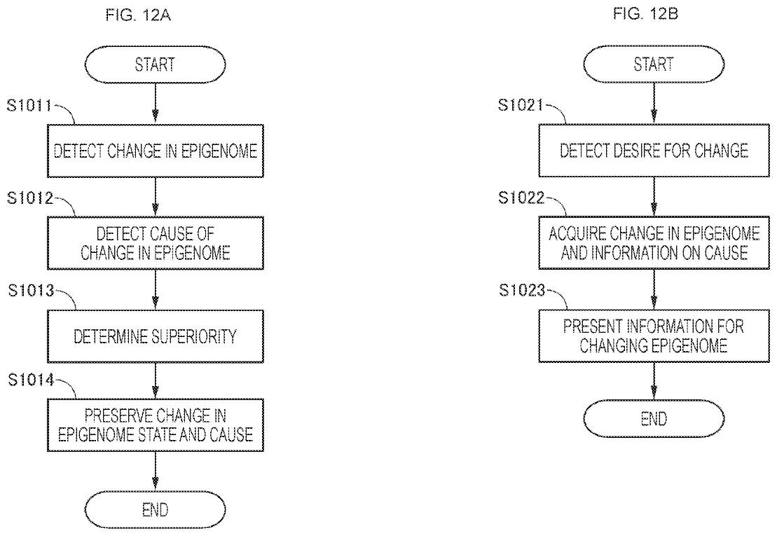

First, a first example will be described with reference to FIG. 12. The information bank is likely to be capable of recognizing at what point in time the epigenome state of the subject has been altered. Furthermore, the information bank is also likely to be capable of recognizing what is the cause of the epigenome change from the lifestyle or action history of the subject. If information regarding many human beings is accumulated, the above likelihood can be verified with statistical superiority. Therefore, it is also considered possible for the information bank to propose, to the subject, an action expected to intentionally cause a change in the epigenome state that is considered to have a good influence.

FIG. 12A depicts processing on the information bank side. As illustrated in FIG. 12A, first, in a case where a change in the epigenome state is detected in step S1011, the information bank detects the cause of the change in the epigenome state in step S1012. Next, in step S1013, the information bank determines statistical superiority on the basis of the gathered information. Then, in step S1014, the information bank preserves the epigenome change and data indicating the cause of the change in the own information bank in association with each other.

Meanwhile, FIG. 12 depicts processing by a user side who uses information from the information bank. As illustrated in the flowchart in FIG. 12B, in the information providing apparatus 100, when detecting a desire of the subject for change such as “I want to alter the situation at present” as illustrated in step S1021, the information providing apparatus 100 acquires, from the information bank, a change in the epigenome and information on the cause for changing the epigenome relevant to the change in the epigenome, in step S1022. Then, in step S1023, the information providing apparatus 100 presents the information on the cause for changing the epigenome to the subject. This makes it possible to propose an action expected to intentionally cause a change in the epigenome to the subject. The personality style can also be improved by the subject aiming the action expected to intentionally cause a change in the epigenome. Then, the information bank acquires epigenome information changed by the action of the subject, whereby the database of the information bank is further enriched.

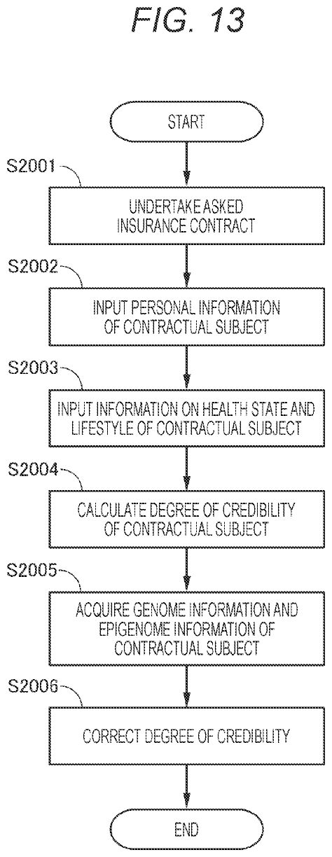

Next, a second example will be described with reference to FIG. 13. In the near future, an insurance company or a bank (referred to as an insurance company or the like) is likely to be allowed to make a contract on the basis of individual's health state, psychological state, purchase history, action pattern, and the like accumulated in an information bank.

First, in a case where an insurance contract is asked for as illustrated in step S2001, a human being who is a subject of the insurance contract (hereinafter referred to as “contractual subject”) is prompted to input personal information to an information bank, as illustrated in step S2002. Next, in step S2003, the contractual subject is prompted to input information on the health state, lifestyle, and the like of the contractual subject to the information bank.

Next, in step S2004, the insurance company or the like calculates the degree of credibility of the contractual subject on the basis of the personal information, the health state, the lifestyle information, and the like of the contractual subject input to the information bank. For example, this degree of credibility is given as a high value in a case where the health state is good and the lifestyle does not have anything that damages health, and is given as a low value in a case where the health state is bad or the lifestyle has something that damages health.

Next, in step S2005, the insurance company or the like acquires the genome information and the epigenome information of the contractual subject from the information bank. Then, in step S2006, the insurance company or the like corrects the individual's degree of credibility on the basis of the genome information and the epigenome information. In this manner, by using the genome information and the epigenome information held in the information bank, inspection and the like in the insurance contract can be performed more precisely for a person wishing the contract. By utilizing the genome information and the epigenome information, it is also possible to evaluate the individual's genetic characteristics and the individual's lifestyle separately. By evaluating in this manner, the credibility in the way of handling the personal information on the information bank side will also increase.

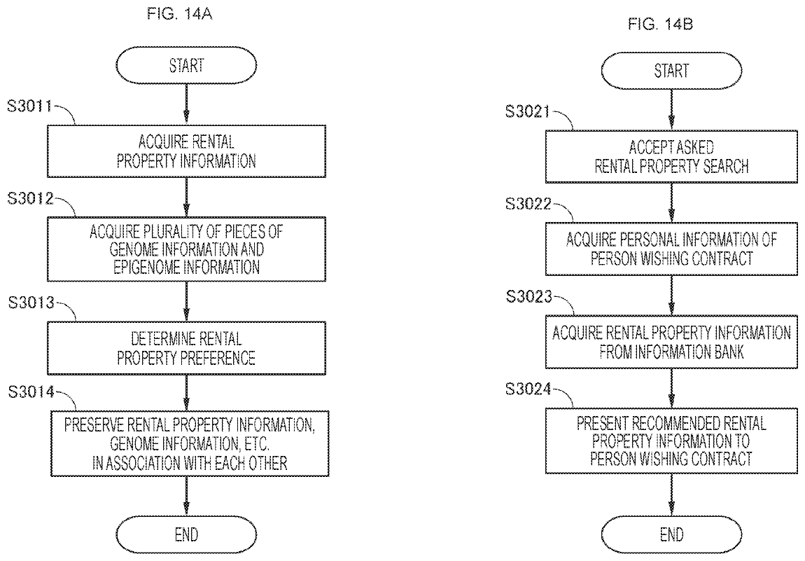

Next, a third example will be described with reference to FIG. 14. The third example is a proposal for a rental property using the genome information and the epigenome information. A real estate agent confirms matching between a request of a person who will make a rental contract (hereinafter referred to as a person wishing the contract) and a rental property on the basis of the genome information and the epigenome information held in the information bank.

First, the information bank acquires rental property information handled by the real estate agent in step S3011 and acquires the genome information and the epigenome information of the persons wishing the contract in step S3012. In step S3013, the information bank determines statistical property preference for the held rental property information. Then, in step S3014, the information bank preserves the rental property information, the genome information, the epigenome information, and a property preference determination result in the own information bank in association with each other.

Meanwhile, as illustrated in step S3021, when accepting an asked rental property search from the person wishing the contract, the real estate agent side acquires the personal information of the person wishing the contract in step S3022. Next, in step S3023, the real estate agent acquires the rental property information from the information bank. Since this rental property information is associated with the genome information, the epigenome information, and the property preference determination result at the information bank, it can be sad that this rental property information is highly likely to match the wish of the person wishing the contract. Then, in step S3024, the real estate agent presents the recommended rental property information acquired from the information bank to the person wishing the contract. By presenting the recommended rental property in this manner, the contract rate can be enhanced. Note that, when the contract is established, a part of the contract fee may be paid from the real estate agent to the information bank operator as a reward.

### 2. Modifications

Although the embodiments of the present technology have been specifically described above, the present technology is not limited to the above-described embodiments, and various modifications based on the technical idea of the present technology are possible.

In the embodiments, it has been described that the genome information, the epigenome information, the life log, the detection information by the sensor, the information indicating the liking and preference, the information obtained from use of the e-mail or the SNS, and the like are held in the information database 300, but the information processing apparatus 200 may retain these pieces of information.

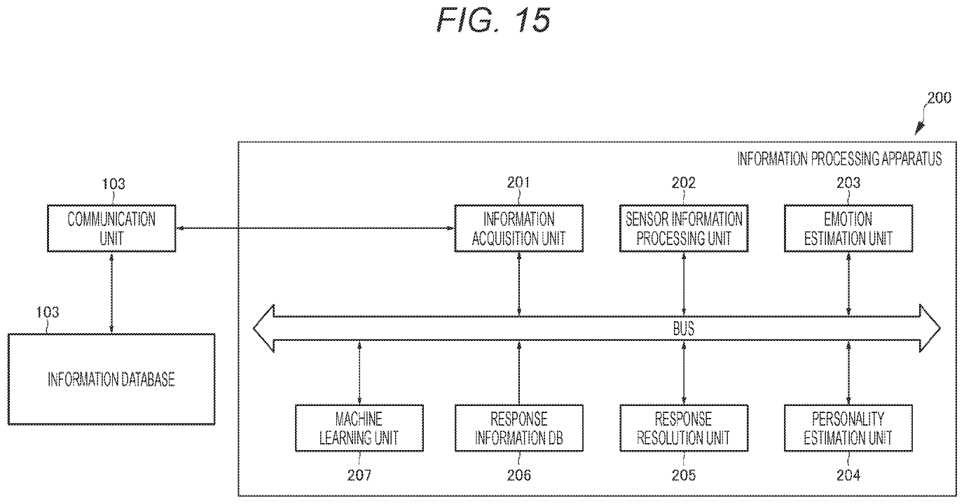

Furthermore, as illustrated in FIG. 15, the information processing apparatus 200 may include a machine learning unit 207, and the emotion estimation unit 203 and the personality estimation unit 204 may be customized by machine learning to raise the estimation accuracy. As a learning method of machine learning, for example, a neural network or deep learning is used. The neural network is a model imitating a human cranial nerve circuit and is made up of three types of layers, namely, an input layer, an intermediate layer (hidden layer), and an output layer. Furthermore, the deep learning is a model using a neural network having a multilayer structure and can learn a complex pattern latent in a large amount of data by repeating learning distinctive for each layer. In addition, as a hardware structure that implements such machine learning, a neurochip or a neuromorphic chip incorporating a concept of a neural network can be used.

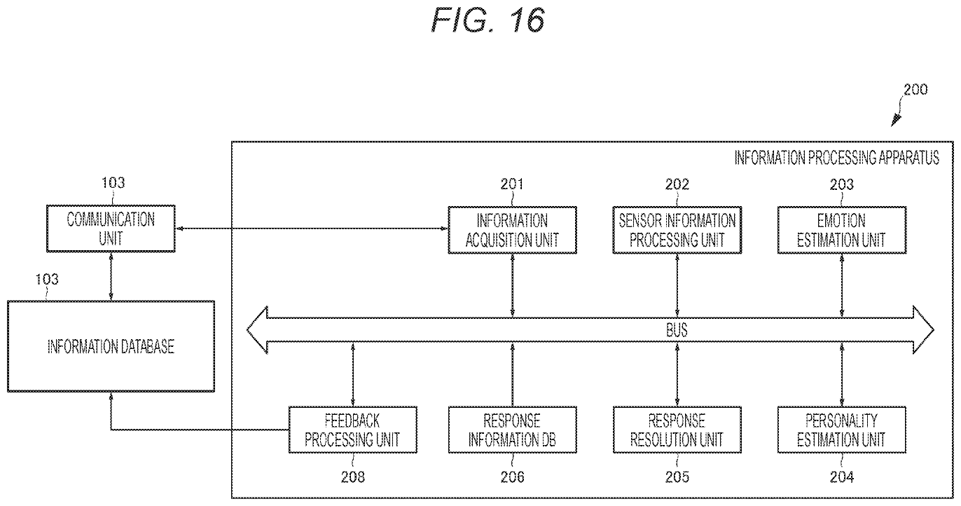

Moreover, as illustrated in FIG. 16, the information processing apparatus 200 may include a feedback processing unit 208 to transmit various sorts of sensor information, the genome information, the epigenome information, and the emotion estimation result and the personality estimation result based on these pieces of information in association with each other to the information database 300 for feedback. The feedback processing unit 208 has a function of aggregating information to be fed back and transmitting the aggregated information to the information database 300. This configuration is considered to enable the exploitation for a variety of use cases and business models. Note that the feedback may be performed by communication via the communication unit 103.

The present technology can be also configured as described below.

(1)

An information processing apparatus including:

an information acquisition unit that acquires genome information and related information regarding a subject; and

an emotion estimation unit that estimates an emotion of the subject on the basis of the genome information and the related information.

(2)

The information processing apparatus according to (1), further including a personality estimation unit that estimates a personality of the subject on the basis of the genome information and the related information.

(3)

The information processing apparatus according to (1) or (2), further including a response resolution unit that resolves a response to the subject on the basis of the emotion of the subject estimated by the emotion estimation unit and the related information.

(4)

The information processing apparatus according to any one of (1) to (3), in which the related information includes epigenome information of the subject.

(5)

The information processing apparatus according to any one of (1) to (4), in which the related information includes biometric information of the subject.

(6)

The information processing apparatus according to any one of (1) to (5), in which the related information includes image information on the subject.

(7)

The information processing apparatus according to any one of (1) to (6), in which the related information includes voice information of the subject.

(8)

The information processing apparatus according to any one of (1) to (7), in which the related information includes action history information of the subject.

(9)

The information processing apparatus according to any one of (1) to (8), in which the related information includes environmental information around the subject.

(10)

The information processing apparatus according to any one of (1) to (9), in which the related information includes sensor information acquired by a sensor.

(11)

The information processing apparatus according to any one of (1) to (10), in which the related information includes history information of exchange between an information providing apparatus in which the information processing apparatus works and the subject.

(12)

The information processing apparatus according to any one of (1) to (11), in which the genome information and/or the related information are/is acquired from an information database that is located externally.

(13)

The information processing apparatus according to (12), in which the emotion of the subject estimated by the emotion estimation unit is fed back to the information database.

(14)

The information processing apparatus according to any one of (1) to (13), in which the emotion estimation unit is updated by machine learning.

(15)

An information processing method including: acquiring genome information and related information regarding a subject; and

estimating an emotion of the subject on the basis of the genome information and the related information.

(16)

An information processing program for causing a computer to execute an information processing method including: acquiring genome information and related information regarding a subject; and

estimating an emotion of the subject on the basis of the genome information and the related information.

### REFERENCE SIGNS LIST

100 Information providing apparatus
200 Information processing apparatus
201 Information acquisition unit
203 Emotion estimation unit
204 Personality estimation unit
300 Information database

1. An information processing apparatus comprising:
an information acquisition unit that acquires genome information and related information regarding a subject; and
an emotion estimation unit that estimates an emotion of the subject on a basis of the genome information and the related information.

2. The information processing apparatus according to claim 1, further comprising
a personality estimation unit that estimates a personality of the subject on a basis of the genome information and the related information.

3. The information processing apparatus according to claim 1, further comprising
a response resolution unit that resolves a response to the subject on a basis of the emotion of the subject estimated by the emotion estimation unit and the related information.

4. The information processing apparatus according to claim 1, wherein
the related information includes epigenome information of the subject.

5. The information processing apparatus according to claim 1, wherein
the related information includes biometric information of the subject.

6. The information processing apparatus according to claim 1, wherein
the related information includes image information on the subject.

7. The information processing apparatus according to claim 1, wherein
the related information includes voice information of the subject.

8. The information processing apparatus according to claim 1, wherein
the related information includes action history information of the subject.

9. The information processing apparatus according to claim 1, wherein
the related information includes environmental information around the subject.

10. The information processing apparatus according to claim 1, wherein
the related information includes sensor information acquired by a sensor.

11. The information processing apparatus according to claim 1, wherein
the related information includes history information of exchange between an information providing apparatus in which the information processing apparatus works and the subject.

12. The information processing apparatus according to claim 1, wherein
the genome information and/or the related information are/is acquired from an information database that is located externally.

13. The information processing apparatus according to claim 12, wherein
the emotion of the subject estimated by the emotion estimation unit is fed back to the information database.

14. The information processing apparatus according to claim 1, wherein
the emotion estimation unit is updated by machine learning.

15. An information processing method comprising:
acquiring genome information and related information regarding a subject; and
estimating an emotion of the subject on a basis of the genome information and the related information.

16. An information processing program for causing a computer to execute an information processing method comprising:
acquiring genome information and related information regarding a subject; and
estimating an emotion of the subject on a basis of the genome information and the related information.
